(19) United States
(12) Patent Application Publication
(10) Pub. No.: US 2005/0148877 A1
(43) Pub. Date: Jul. 7, 2005

(54) Multidimensional transducer probe with different transmit and receive segments for medical ultrasound imaging

(75) Inventors: Xiaocong Guo, Woodinville, WA (US); Todor Sheljaskow, Issaquah, WA (US); Linxin Yao, Bellevue, WA (US); Grazyna Palczewska, Bellevue, WA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/738,685

(22) Filed: Dec. 17, 2003

(51) Int. Cl. A61B 8/14

(52) U.S. Cl. 600/459

(57) ABSTRACT

Methods, systems, and probes are provided for medical ultrasound imaging. By using larger segments for transmit than receive or by using a sparse sampling of elements on transmit than used for receive, the number of transmit beamformer channels relative to receive beamformer channels is reduced. Where the transmit waveformed generators of the transmit beamformer channels are positioned within an ultrasound probe, the space and power requirements of the transmit beamformer channels are reduced based on the reduction in number of transmit segments. Different approaches may be used for reducing the number of transmit channels relative to receive segments. For example, a flexible circuit is connected to one side of a multi-dimensional array and defines transmit segments as groups of two or more elements. A different flexible circuit connects on an opposite side of the elements. The different flexible circuit defines receive segments as individual elements or a fewer number of elements than the transmit segments. Alternatively, switching is used to combine elements into single transmit segments. As another alternative, switching or configuring electrode circuits is used to define transmit segments as sparsely-spaced elements and the receive segments as a less sparsely spaced. In yet another approach, the transmit aperture covers a lesser area than the receive aperture, such as through sparse spacing or having smaller overall dimensions.

### BACKGROUND

The present invention relates to transducer arrays, such as multi-dimensional transducer arrays. In particular, multi-dimensional transducer arrays for medical diagnostic imaging are provided.

Ultrasound transducers connect through cables with imaging systems. For linear arrays, 64, 128 or 256 elements connect through separate cables to the imaging system. A similar number of transmit and beamformer channels are provided for generating acoustic transmit beams and receiving samples of a scanned region. To avoid electrical cross-talk and other interference, coaxial cables are used for communicating from the transducer array to the imaging system. As the number of elements increases, the number of coaxial cables increases. However, miniaturization of coaxial cables is expensive and limited.

For multi-dimensional arrays, such as two-dimensional arrays, the number of elements may be drastically increased as compared to one-dimensional arrays. A corresponding increase in the number of transmit and receive beamformers channels is expensive and results in bulky or unusable cables. One approach to limit the number of cables is to multiplex received signals from different elements as a function of time onto a fewer number of cables than elements. However, multiplexing may be inappropriate for or not used for transmit signals. Another approach includes using one set of elements, such as a grouping of elements sparsely distributed on the array for transmit and a different set of elements for receive operations. A fewer number of transmit elements are provided, resulting in a fewer number of transmit beamformer channels. However, providing separate transmit and receive elements may adversely affect the received signals and require extra elements.

### BRIEF SUMMARY

By way of introduction, the preferred embodiments described below include methods, systems, and probes for medical ultrasound imaging. By using larger segments for transmit than receive or by using a sparse sampling of elements on transmit than used for receive, the number of transmit beamformer channels relative to receive beamformer channels is reduced. Where the transmit waveformed generators of the transmit beamformer channels are positioned within an ultrasound probe, the space and power requirements of the transmit beamformer channels are reduced based on the reduction in number of transmit segments.

Different approaches may be used for reducing the number of transmit channels relative to receive segments. For example, a flexible circuit is connected to one side of a multi-dimensional array and defines transmit segments as groups of two or more elements. A different flexible circuit connects on an opposite side of the elements. The different flexible circuit defines receive segments as individual elements or a fewer number of elements than the transmit segments. Alternatively, switching is used to combine elements into single transmit segments. As another alternative, switching or configuring electrode circuits is used to define transmit segments as sparsely-spaced elements and the receive segments as a less sparsely spaced. In yet another approach, the transmit aperture covers a lesser area than the receive aperture, such as through sparse spacing or having smaller overall dimensions.

In a first aspect, a multi-dimensional transducer array is provided for medical ultrasound imaging. A plurality of elements is spaced in a multi-dimensional grid. A structure operable to form a first segment with at least a first element of a plurality of elements is also operable to form a second segment with the first element and an additional element of the plurality. The first segment is free of the additional element.

In a second aspect, a multi-dimension transducer system is provided for medical ultrasound imaging. An array of elements is in a multi-dimensional grid pattern. Transmit beamformer channels are connectable with the array. Receive beamformer channels are also connectable with the array. A fewer number of transmit beamformer channels are used for a transmit aperture than receive beamformer channels used for a receive aperture. The transmit and receive apertures have some of the elements in common.

In a third aspect, a method for reducing transmit channels is provided for multi-dimensional transducer arrays. A transmit aperture is formed with a plurality, N, of transmit segments on the multi-dimensional transducer array. A receive aperture is formed having a plurality, M, of receive segments on the multi-dimensional transducer array. N is less than M, and the transmit aperture includes a plurality of elements of the array also included in the receive aperture.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed on illustrating the principles of the invention. Moreover, in the figures, like reference numerals designate corresponding parts throughout the different views.

FIG. 1 is a block diagram of one embodiment of a multi-dimensional transducer array system;

FIG. 2 is a top view of a cutaway portion of a multi-dimensional transducer array in one embodiment; and

FIG. 3 is a flow chart diagram of one embodiment of a method for reducing transmit channels in a multi-dimensional transducer array.

### DETAILED DESCRIPTION OF THE PRESENTLY PREFERRED EMBODIMENTS

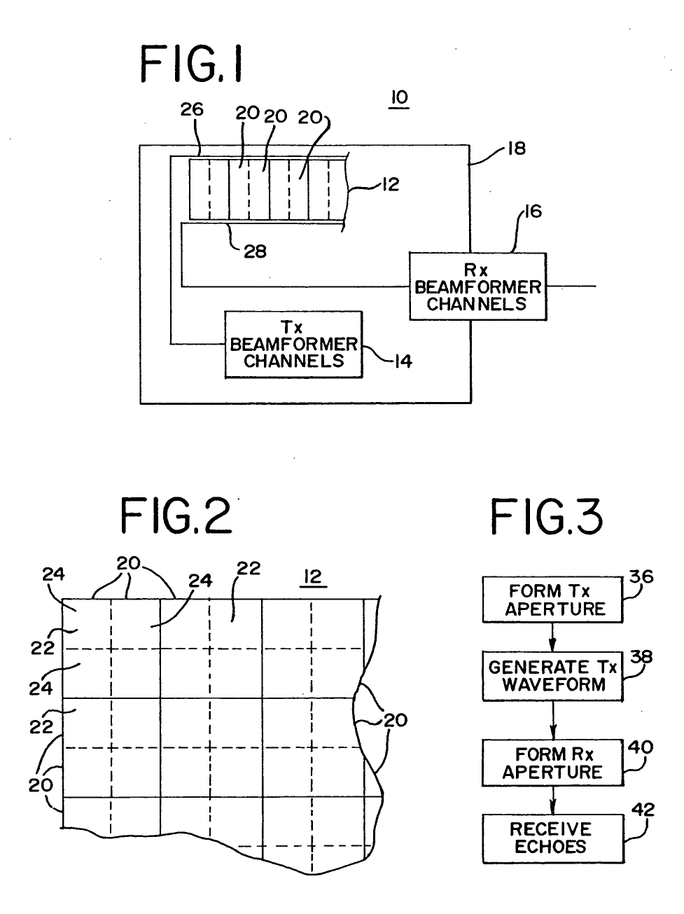

FIG. 1 shows a multi-dimensional transducer system 10 for medical ultrasound imaging in one embodiment. The system 10 includes an array 12 of elements, transmit beamformer channels 14, and receive beamformer channels 16. As shown, the array 12 of elements, the transmit beamformer channels 14 and a portion of the receive beamformer channels 16 are positioned within a probe housing 18. In alternative embodiments, the transmit beamformer channels 14 and/or receive beamformer channels 16 are positioned in an imaging system and connect with the array 12 of elements through a cable. The transmit beamformer channels 14 and receive beamformer channels 16 are shown connected to the array 12 of elements on separate paths or circuits. In alternative embodiments, the transmit beamformer channels 14 and receive beamformer channels 16 connect to the array 12 using a same path through a transmit/receive switch.

The array 12 includes a plurality of elements 20 positioned in a multi-dimensional grid pattern. As shown in FIG. 2, the multi-dimensional grid pattern in one embodiment has elements 20 space along a plurality of columns and rows. In the embodiment shown in FIG. 2, each of the elements 20 is spaced along the grid pattern with half-wavelength spacing. A desired imaging frequency is used to determine the distance from the center of each element 20 to adjacent elements. Greater or lesser wavelength spacings may be provided. Each of the elements 20 are separated by a kerf or other electrical and/or physical separation. In one embodiment, the multi-dimensional grid pattern is a two-dimensional grid pattern, but any rectangular, square, hexagonal or other shaped grids may be used. For example, the array 12 is a 1.25, 1.5, 1.75 or 2-dimensional array. As another example, the array 12 has one or more rows or columns with fewer elements 20 than other rows or columns.

Each of the elements 20 is a piezoelectric or capacitive membrane-based transducer of acoustic and electrical energies. Alternatively, other now-known or later-developed materials or structures for transducing between acoustic and electrical energy may be used. In one embodiment, each of the elements 20 is a composite material of piezoelectric and a polymer, silicon, rubber or other bonding material for holding posts or fragments of the piezoelectric in positions relative to each other. The kerfs separating each of the elements 20 are filled with air, rubber, silicon, polymer or other now-know or later-developed material.

The probe housing 18 is plastic, rubber, metal or now-known or later-developed material for at least partially housing the array 12. In one embodiment, the probe housing 18 is adapted for handheld use, such as being shaped to have a grip or other portion for holding by the user. Alternatively, the housing 18 is shaped for use internally in a patient, such as an endoscope or catheter device. The probe housing 18 includes an acoustic window, such as a polymer, plastic, glass or air window for allowing transmission of acoustic energy from the array 12 into a patient. In one embodiment, the probe housing 18 or the system 10 use the components, structure or materials disclosed in U.S. Pat. No. ______ (application Ser. No. 10/184,785), the disclosure of which is incorporated herein by reference. For example, the transmit beamformer channels 14 and receive beamformer channels 16 of the system 10 of FIG. 1 use the transmit and receive beamformer structures disclosed in the above-referenced patent. The position of the transmit beamformer channels 14 and receive beamformer channels 16 relative to the probe housing 18 are also the same as in the above-referenced patent. In alternative embodiments, the position of the components, the components, or other aspects are different.

The transmit beamformer channels 14 are electrical traces connecting between a transmit waveformed generator and the array 12. In other embodiments, the transmit beamformer channels 14 include delays, timing circuits, amplifiers, waveform generators or combinations thereof for generating relatively delayed and apodized waveforms for each of a plurality of transmit segments on the array 12. In one embodiment, the transmit beamformer channels 14 are connectable with the array 12, such as by having direct, permanent electrical connections or through a transmit and receive or other switch. In one embodiment, the transmit beamformer channels 14 are implemented on one or more application-specific integrated circuits, processors, field-programmable gate arrays, digital circuits, analog circuits, combinations thereof or other now-known or later-developed devices within the probe housing 18. Alternatively, a portion or all of the transmit beamformer channels 14 other than the traces or signal lines are positioned outside of the transducer probe housing 18. In one embodiment, the waveform generators are transistors, networks or other devices for generating unipolar or bipolar waveforms.

Each of the transmit beamformer channels 14 connects with different transmit segments. All of the transmit segments together form a transmit aperture. Different relatively delayed or apodized waveforms are applied to different transmit segments for generating a beam, fan or other distribution of acoustic energy with the array 12. Each transmit segment is formed with one or more elements 20. As used herein, a segment is defined by the electrical connections, electrodes or elements 20 responsive to a same transmit beamformer channel 14 or separately electrically addressable connection. Similarly, receive segments are responsive to or provide information to a same receive beamformer channel or separate electrical connection.

The receive beamformer channels 16 are signal traces, amplifiers, delays, summers, multipliers, phase rotators, digital circuits, analog circuits, combinations thereof or other now-known or later-developed receive beamformer channels. In one embodiment, each receive beamformer channel 16 within the probe housing 18 includes signal traces to different receive segments with or without a multiplexer or other switching, preamplifiers and a multiplexer for applying time-division multiplexing to a plurality of receive beamformer channels 16. Alternatively, one or more summers for partial or complete beamforming are within the probe housing. The signals from the plurality of channels are multiplexed onto a same signal line for later demultiplexing and beamforming. Additional, or fewer components of the receive beamformer channels 16 may be included within the transducer probe housing 18. As used herein, a receive beamformer channel 16 may include only receive signal lines for outputting data to a receive beamformer. Likewise, a transmit beamformer channel 14 may include only signal lines for connection with a transmit beamformer. Each of the receive beamformer channels 16 or a subset of the channels are connected or connectable with different receive segments.

A fewer number of transmit beamformers channels 14 are used for the transmit aperture than receive beamformer channels 16 used for receive aperture. To minimize the spatial requirements of the array 12, the transmit and receive apertures have at least some or all of the elements 20 in common. By reducing the number of transmit segments and associated transmit beamformer channels 14, fewer components and space are used for transmit beamformer channels 14 within the probe housing 18. In one embodiment, the number of transmit beamformer channels 14 and associated transmit segments are fewer by a multiple of 2, 4, other integer number or a non-integer number than the number of receive beamformer channels 16 and associated receive segments. For example, four receive segments and associated beamformer channels 16 are provided for each transmit segment and associated beamformer channel 14. As another example, the transmit aperture includes M segments connectable with M transmit beamformer channels. The receive aperture includes N segments connectable with N receive beamformer channels. N is greater than M. A fewer number of segments are used during a transmit event than are used during a receive event. At least one element 20 used with a segment in the receive aperture is also used with a segment of the transmit aperture.

In one embodiment, the difference in number of transmit and receive segments is provided by using larger transmit segments of the transmit aperture than receive segments of the receive aperture. The transmit aperture is a same, larger or lesser area and/or location than the receive aperture. For example, a structure is operable to form a receive segment using at least one element 20 and operable to form a transmit segment using the same element 20 and an additional element 20. The additional element is not used with the same receive segment as the first element 20. For example, at least two elements 20 are used for each separate transmit segment. Different pairs of elements form different transmit segments. For receive segments, each receive segment is formed from a fewer number of the elements 20. For example and referring to FIG. 2, the solid lines separate a plurality of transmit segments 22. Each of the transmit segments 22 includes four elements 20, but a fewer or greater number of elements 20 may be used for a given transmit segment 22. Each transmit segment 22 is square in shape, but other rectangular, or irregular shapes may be used. Each receive segment is configured as a single element 20. The dashed lines represent separation of the elements 20 and the receive segments 24. The dashed and solid lines represent the separation of the elements 20. The receive segments 24 are made of single ones of the elements 20, and the transmit segments 22 are made of a multiple of four of the elements 20. The same element 20 is used for both the transmit segment 22 and the receive segment 24. Alternatively, one or more elements 20 are used for only a transmit or only a receive segment 22, 24. In the example given above with half-wavelength spacing between each of the elements 20, the transmit segment 22 has a one-wavelength spacing in the grid of the array 12 between each of the plurality of transmit segments 22. The receive segments 24 have a one-half wavelength spacing within the grid. Other multiples than four may be used. In other embodiments, different transmit segments 22 or receive segments 24 use different numbers of elements 20. In yet other embodiments, the receive segments 24 include two or more elements 20.

In one embodiment, the structure is a pattern of electrodes forming transmit segments and a different pattern of electrodes forming receive segments. For example, the transmit electrodes cover multiple elements 20 on one side of the array 22, such as a top side. The electrodes for the receive segments are on an opposite side of the array 12, such as the bottom side. The electrodes comprise conductive material, such as copper, nickel-plated copper, gold or other now-known or later-developed conductive material.

In one embodiment, a flexible circuit 26 has electrical traces and other conductive deposits defining the transmit segments of the transmit aperture. The flexible circuit 26 is bound with epoxy, pressure or other material or structure to a top side of the array 12, but may be bound to a bottom side in alternative embodiments. Similarly, a flexible circuit 28 has a plurality of electrical traces and associated conductive deposits defining receive segments of the receive aperture on a bottom side of the array 12. Alternatively, the receive flex circuit is positioned on a top of the array 12. For a dense distribution of receive segments 24 and/or elements 20, the flexible circuit 28 for the receive segments may include a multiple layer flexible circuit. A multi-layer flexible circuit allows a greater density of signal traces. Using vias or other through-hole technology, the different traces connect to different elements 20 or conductive paths defining electrodes for each of the receive segments. Since the transmit segment density may be less, a single-sided or layer flex circuit may be used for the transmit aperture. Alternatively, a multiple-layer flexible circuit 26 is used for the transmit aperture. Where a single layer is used, a grounding plane may be formed on another side of the flexible circuit, such as above the array 12 for use during receive operations. Using different flexible circuits for transmit and receive segments on opposite sides of the array 12 and associated elements 20 allows for electrical separation between the transmit channels 14 and the receive beamformer channels 16 without a transmit receive switch, such as disclosed in the above-referenced U.S. Pat. No. ______ (Ser. No. 10/184,785).

In alternative embodiments, the structure includes a plurality of switches. For example, a multiplexer is operable to electrically connect and disconnect electrodes associated with each of the elements 20 to different ones of the transmit beamformer channels 14 and receive beamformer channels 16. Using switching, a same flexible circuit or traces may be used for both transmit and receive operations. The top of the array 12 has a grounding plane without separate segment traces, but separate segment traces may be used. A multi-layer flexible circuit or other structure independently addresses each of the elements 20. The multiplexer is used to switch together multiple of the elements 20 to a same transmit beamformer channel 14. Separate elements connect with receive beamformer channels 16.

In the embodiments discussed above, the transmit and receive apertures have a same area, such as using all of the same elements 20. In alternative embodiments, the same area is provided for each of the transmit and receive apertures, but with some different elements 20. For example, the transmit aperture may be shifted or include different sparse sampling than the receive aperture. At least some overlap of the transmit and receive apertures is provided.

In an alternative embodiment, the number of transmit beamformer channels 14 is reduced through a different transmit aperture size. Using a same size or different size transmit segments as receive segments, a lesser area for the transmit aperture uses fewer transmit beamformer channels than a larger area receive aperture uses receive beamformer channels. For example, a transmit aperture of 400 segments spaced at a center of an array 12 uses transmit segments each comprising a single element 20. The receive aperture covers the entire extent or a larger extent of the array 12, such as using receive segments for each element 20 with a total of 1,600 or other number of segments. As another example, the transmit aperture has a more sparse sampling than the receive aperture. For example, the same spatial extent or a different spatial extent is provided for the transmit aperture than the receive aperture, but with a less dense sampling of elements 20 for the transmit aperture. As shown in FIG. 2, an example sparse transmit aperture uses the lower right or other single element 20 of each set of four elements as a transmit segment. The other three elements 20 of this set are not used for transmit operation but are used as separate segments for receive operation. Different spatial distributions may be provided for sparse sampling of the transmit array using segments of a single or a multiple elements 20.

Using a same or similar size transmit aperture as receive aperture free of sparse sampling may provide advantages. Where each of the transmit and receive apertures are a same size or use the entire array 12, better signal to noise and lateral resolution may be provided. For tissue or contrast agent harmonic imaging, the acoustic energy is transmitted at about half of the center frequency of the transducer array 12 (e.g., 2 MHz). The half of the center frequency used on transmit corresponds to a half-wavelength sampling of transmit segments using four elements 20. During receive operation, a half-wavelength sampling between receive segments is provided using individual ones of the elements 20. The second harmonic (e.g., 4 MHz) of the transmitted fundamental frequency corresponds to half-wave length sampling of the receive segments. As a result, the clutter level and grating lobe interference may be reduced. Other harmonics, including fractional or sub-harmonics may be used. For sub-harmonics, the transmit segments may be smaller than the receive segments. For fundamental imaging, an imaging frequency is selected in between the half-wavelength spacing of the transmit segments and the half-wavelength spacing of the receive segments. Alternatively, the imaging frequency is selected independent of the wavelength spacing or based on the wavelength spacing of either the transmit segments 22 or the receive segments 24. Since a grating lobe may be at different angles given the different spacing of the transmit segments 22 and the receive segments 24, the resulting two-way grating lobe interference may be minimal. Other advantages, different advantages, only one of the advantages discussed above or none of the advantages discussed above may be provided.

FIG. 3 shows one embodiment of a method for reducing transmit channels in a multi-dimensional transducer array. The method of FIG. 3 is implemented using the structure or components of FIG. 1 or 2 in one embodiment, but different structures or components may be used in other embodiments. The number of transmit beamformer channels and associated transmit segments is less than the number of receive beamformer channels and associated receive segments. For example, the number of receive segments is at least double or quadruple the number of transmit segments. The transmit aperture includes at least one, a plurality or all of the elements also included in the receive aperture. Any of various approaches may be used for the difference in the number of transmit and receive segments. For example, the transmit segments are larger than the receive segments. As another example, the transmit segments are more sparse than the receive segments. As yet another example, the transmit aperture is smaller than the receive aperture.

In act 36, a transmit aperture is formed. The transmit aperture has a plurality, N, of transmit segments on a multi-dimensional transducer array. In one embodiment, one or more of the transmit segments includes a plurality of elements. Groups of elements are connected together to form each of the transmit segments. The connection is provided as part of the physical structure, such as an electrode or flexible circuit electrically connected together. The electrode pattern defines the transmit segments. Alternatively, a multiplexing or other switching structure allows selective connection of different elements to form each transmit segment. Using either the switching or pattern structure, a transmit beamformer channel is connected to at least two or more elements for each transmit segment. Different elements are used for different segments.

In act 38, transmit waveforms are generated. A separate waveform is provided for each transmit segment of the transmit aperture. The waveforms may be different, such as associated with different apodization weighting or relative delays, but may be the same. In response to application of the waveforms to different transmit segments, a transmit beam or fan of acoustic energy is generated by the transducer array. In one embodiment, the transmit waveforms are generated for the transmit aperture in a probe. For example, transmit waveform generators are provided in a handheld transducer probe with the array. Alternatively, the transmit waveforms are generated in an imaging system and provided through one or more cables to the array.

In act 40, a receive aperture is formed. The receive aperture has a plurality, M, of receive segments on the multi-dimensional transducer array. The number, M, of receive segments is fewer than the number, N, of transmit segments. Various approaches are used to have more receive segments than transmit segments. For example, none or a fewer number of elements are connected together to form receive segments than are connected together for transmit segments. Only one element is used for each receive segment in one embodiment. As a result, each receive beamformer channel connects to a fewer number of the elements than a corresponding transmit beamformer channel. A same element may be used for both transmit and receive segments while a different element is used for a different receive segment but a same transmit segment.

In act 42, acoustic echoes are received by the array using the receive segments. Electrical signals generated by the elements in response to the acoustic echoes are transmitted from the electrodes to the receive beamformer channels.

The array 12 shown in FIGS. 1 and 2 may be manufactured using any possible technique. In one embodiment, the method of manufacture disclosed in U.S. Pat. No. ______ (application Ser. No. 10/184,785) is altered due to the different size of the transmit segments 22. Since the flexible circuit used for the transmit aperture has fewer total segments, such as one-fourth fewer total segments, than the receive aperture, a single flex circuit may be provided across the top of the array for operating with elements corresponding to a plurality, such as four, flexible circuits for the receive segments. The array 12 is formed out of four modules in one example embodiment where each module has its own receive flex circuit. A transmit aperture flexible circuit is then applied in common to all of the array or modules. Other number of modules may be used in other embodiments.

For example, the bottom flexible circuits for the receive aperture, backing, element materials and matching layers are laminated. Dicing cuts then form kerfs along the azimuth and elevation dimensions. The lamination and dicing are repeated for each of the modules, such as four modules with a 0.330 millimeter pitch. The modules are then assembled together. The dicing cuts or kerfs are used to align the modules, but other alignment techniques may be used. The space between the modules is minimized since only a single flex circuit is provided on one or two sides of the backing block between modules. The top flexible circuit is then applied to the multi-module assembly. Since the number of transmit segments is reduced as compared to the number of receive segments, the density of signal traces on the top flexible circuit may be less, allowing a single flexible circuit for multiple modules. In alternative embodiments, multiple flexible circuits for each module or for groups of modules of an array are provided for the transmit aperture. In yet other alternative embodiments, the transmit apertures define on a bottom of the array 12 and the receive aperture is formed on the top of the array.

In another embodiment, the backing and flexible circuits of each module are laminated together. Feducials are then used to align the modules together for assembly. The feducials are provided on a flexible circuit and lamination tool. The ceramic and matching layers are then laminated onto the multi-module structure and diced along the elevation and azimuth dimensions. As discussed above, the flexible circuit for the transmit aperture is then applied over multiple modules associated with different receive segment flexible circuits.

In an alternative method of manufacture, no modules or a fewer number of modules are used for forming the multi-dimensional array. The flexible circuit for the receive aperture is a multiple layer flexible circuit. Multiple layers allow for an increase in the density of traces. Using vias or other connection technologies, each of the traces are connected with an electrode or different elements of the receive segments. Since the transmit aperture flexible circuit has a lesser density, a single layer is used. Multiple layer flexible circuits may be used in alternative embodiments for the transmit aperture. The transducer is then assembled using now-known or later-developed stacking, dicing, lamination, bonding, alignment or other acts.

For transmit operation using different flexible circuits on different sides of the transducer array, the receive flexible circuit or beamformer channels are grounded using diodes or other devices. For receive operation in this structure, the transmit beamformer channels are grounded using switches or other devices. In one embodiment, a bottom of the flexible circuit for the transmit aperture is used to define the segments. The top of the flexible circuit is coated or formed with a ground plane. The single ground plane is then switched to ground without requiring a switch for each transmit beamformer channel. During transmit operations, the ground plane on the upper side of the flexible circuit is allowed to float or is an open circuit from ground. Alternatively, the flexible circuit Kapton or other material provides a dielectric insulator and the ground plane is maintained as the permanent connection to ground.

While the invention has been described above by reference to various embodiments, it should be understood that many changes and modifications can be made without departing from the scope of the invention. It is therefore intended that the foregoing detailed description be regarded as illustrative rather than limiting, and that it is the following claims, including all equivalents, that are intended to define the spirit and the scope of this invention.

1. A multi-dimensional transducer array for medical ultrasound imaging, the transducer comprising:
a plurality of elements spaced in a multi-dimensional grid; and
a structure operable to form a first segment with at least a first element of the plurality of elements and operable to form a second segment with at least the first element and a second element of the plurality of elements, the first segment free of the second element.

2. The transducer array of claim 1 wherein the structure comprises a first electrode forming the first segment and a second electrode forming the second segment.

3. The transducer array of claim 2 wherein the first electrode is on one side of the first element and the second electrode is on an opposite side of the first element.

4. The transducer array of claim 1 wherein the structure comprises a switch operable to electrically connect and disconnect a first electrode of the first element with a second electrode of the second element.

5. The transducer array of claim 1 wherein the second segment comprises four of the plurality of elements including the first element and the first segment comprises a single one of the plurality of elements, the single one being the first element.

6. The transducer array of claim 1 further comprising:
a plurality of transmit segments including the second segment, each transmit segment formed from at least two of the plurality of elements, each transmit segment having different elements than the other transmit segments; and
a plurality of receive segments including the first segment, each receive segment formed from fewer of the plurality of elements than the transmit segments, the receive segments formed from the elements used for the transmit segments.

7. The transducer array of claim 5 wherein the second segment has a wavelength spacing in a grid of a plurality of different second segments and the first segment has a half wavelength spacing in a grid of a plurality of different first segments.

8. The transducer array of claim 1 further comprising:
transmit beamformer channels, one of the transmit beamformer channels connectable with the second segment; and
receive beamformer channels, one of the receive beamformer channels connectable with the first segment;
wherein a transmit aperture including the second segment is operable to connect with the transmit beamformer channels during a transmit event, the transmit aperture including M segments connectable with M transmit beamformer channels; and
wherein a receive aperture including the first segment is operable to connect with the receive beamformer channels during a receive event, the receive beamformer including N segments connectable with N receive beamformer channels, N greater than M.

9. The transducer array of claim 1 wherein the structure comprises a multiple layer flexible circuit positioned on a bottom side of the plurality of elements, the multiple layer flexible circuit operable to form the first segment and wherein the structure comprises a second flexible circuit positioned on a top side of the plurality of elements, the second flexible circuit operable to form the second segment.

10. A multi-dimensional transducer system for medical ultrasound imaging, the system comprising:
an array of elements in a multidimensional grid pattern;
transmit beamformer channels connectable with the array; and
receive beamformer channels connectable with the array;
wherein a fewer number of transmit beamformer channels are used for a transmit aperture than receive beamformer channels used for a receive aperture, the transmit and receive apertures having some of the elements in common.

11. The system of claim 10 wherein the transmit aperture comprises a smaller aperture than the receive aperture.

12. The system of claim 10 wherein the transmit aperture is more sparse than the receive aperture.

13. The system of claim 10 wherein transmit segments of the transmit aperture are larger than receive segments of the receive aperture.

14. The system of claim 13 wherein the transmit segments comprise multiple of the elements and the receive segments comprise single ones of the elements.

15. The system of claim 10 further comprising:
a first flexible circuit having electrical traces defining transmit segments of the transmit aperture; and
a second flexible circuit having electrical traces defining receive segments of the receive aperture.

16. The system of claim 15 wherein the first flexible circuit is on a top side of the array and the second flexible circuit is on a bottom side of the array.

17. The system of claim 10 wherein there are fewer transmit beamformer channels and associated transmit segments by at least a multiple of two than receive beamformer channels and associated receive segments.

18. The system of claim 17 wherein there are fewer transmit beamformer channels and associated transmit segments by at least a multiple of four than receive beamformer channels and associated receive segments.

19. The system of claim 10 further comprising:
a probe housing at least partially around the array;
wherein the transmit beamformer channels comprise respective waveform generators with the probe housing.

20. A method for reducing transmit channels in a multidimensional transducer array, the method comprising:
(a) forming a transmit aperture having a plurality, N, of transmit segments on the multidimensional transducer array; and
(b) forming a receive aperture having a plurality, M, of receive segments on the multidimensional transducer array;
wherein N is less than M and the transmit aperture includes a plurality of elements of the array also included in the receive aperture.

21. The method of claim 20 wherein (a) comprises connecting together groups of the plurality of elements for each of the transmit segments, and wherein (b) comprises connecting together one of: none and fewer of the plurality of element for each of the receive segments.

22. The method of claim 20 wherein (a) comprises connecting a transmit beamformer channel to at least two of the plurality of elements, the at least two being one of the transmit segments, and wherein (b) comprises connecting a receive beamformer channel to a fewer number of the plurality of elements than the transmit beamformer channel, the fewer number being one of the receive segments, the one of the transmit segments and the one of the receive segments having at least one element in common.

23. The method of claim 20 wherein (a) comprises positioning a first electrode pattern on a first side of the plurality of elements, the first electrode pattern defining the transmit segments, and wherein (b) comprises positioning a second electrode pattern on a second side of the plurality of elements, the second electrode pattern defining the receive segments.

24. The method of claim 20 wherein the transmit segments are larger than the receive segments.

25. The method of claim 20 wherein the transmit segments are more sparse than the receive segments.

26. The method of claim 20 wherein the transmit aperture is smaller than the receive aperture.

27. The method of claim 20 further comprising:
(c) generating transmit waveforms for the transmit aperture in a probe, the probe at least partially housing the plurality of elements.

28. The method of claim 20 wherein M is at least double N.

29. The method of claim 20 wherein M is at least quadruple N.

30. The method of claim 20 further comprising:
(c) transmitting at a fundamental frequency with the transmit aperture; and
(d) receiving at a harmonic of the fundamental frequency with the receive aperture.

31. The transducer array of claim 1 further comprising:
transmit beamformer channels, one of the transmit beamformer channels connectable with the first segment for transmission at a fundamental frequency; and
receive beamformer channels, one of the receive beamformer channels connectable with the second segment for reception at a sub-harmonic frequency;
wherein a transmit aperture including the first segment is operable to connect with the transmit beamformer channels during a transmit event; and
wherein a receive aperture including the second segment is operable to connect with the receive beamformer channels during a receive event.

32. The transducer array of claim 1 further comprising:
transmit beamformer channels, one of the transmit beamformer channels connectable with the second segment for transmission at a fundamental frequency; and
receive beamformer channels, one of the receive beamformer channels connectable with the first segment for reception at a harmonic frequency of the fundamental frequency;
wherein a transmit aperture including the second segment is operable to connect with the transmit beamformer channels during a transmit event; and
wherein a receive aperture including the first segment is operable to connect with the receive beamformer channels during a receive event.
